(19) United States
(12) Patent Application Publication
(10) Pub. No.: US 2015/0018913 A1
(43) Pub. Date: Jan. 15, 2015

(54) LEADS, SYSTEMS, AND METHODS FOR NEUROMODULATION USING SUPERPOSITION SIGNALS

(71) Applicant: BOSTON SCIENTIFIC NEUROMODULATION CORPORATION, Valencia, CA (US)

(72) Inventor: Jacob B. Leven, Huntington Beach, CA (US)

(21) Appl. No.: 14/325,176

(22) Filed: Jul. 7, 2014

Related U.S. Application Data
(60) Provisional application No. 61/845,021, filed on Jul. 11, 2013.

(51) Int. Cl. A61N 1/05

(52) U.S. Cl. CPC A61N 1/05
USPC 607/116

(57) ABSTRACT

An electrical stimulation lead includes at least one lead body having a distal end portion, a proximal end portion, and a longitudinal length; multiple electrodes disposed along the distal end portion of the at least one lead body; and at least one terminal disposed along the proximal end portion of the at least one lead body. The lead has more electrodes than terminals. The lead also includes a signal separator disposed along the lead between the electrodes and the at least one terminal; at least one terminal conductor electrically coupling the at least one terminal to the signal separator; and multiple electrode conductors. Each electrode conductor electrically couples the signal separator to a different one of the electrodes. The lead has more electrode conductors than terminal conductors. The signal separator receives signals from the at least one terminal conductor and separates the signals by frequency into electrode signals.

### CROSS-REFERENCE TO RELATED APPLICATIONS

This application claims the benefit under 35 U.S.C. §119(e) of U.S. Provisional Patent Application Ser. No. 61/845,021, filed Jul. 11, 2013, which is incorporated herein by reference.

### FIELD

The present invention is directed to the area of implantable electrical stimulation systems and methods of making and using the systems. The present invention is also directed implantable electrical stimulation leads using superposition signals for delivering neuromodulation signals, as well as methods of making and using the leads and electrical stimulation systems.

### BACKGROUND

Implantable electrical stimulation systems have proven therapeutic in a variety of diseases and disorders. For example, spinal cord stimulation systems have been used as a therapeutic modality for the treatment of chronic pain syndromes. Peripheral nerve stimulation has been used to treat chronic pain syndrome and incontinence, with a number of other applications under investigation. Functional electrical stimulation systems have been applied to restore some functionality to paralyzed extremities in spinal cord injury patients.

Stimulators have been developed to provide therapy for a variety of treatments. A stimulator can include an implantable pulse generator (IPG), one or more leads, and an array of stimulator electrodes on each lead. The stimulator electrodes are in contact with or near the nerves, muscles, or other tissue to be stimulated. The pulse generator generates electrical pulses that are delivered by the electrodes to body tissue.

### BRIEF SUMMARY

In one embodiment, an electrical stimulation lead includes at least one lead body having a distal end portion, a proximal end portion, and a longitudinal length; multiple electrodes disposed along the distal end portion of the at least one lead body; and at least one terminal disposed along the proximal end portion of the at least one lead body. The lead has more electrodes than terminals. The lead also includes a signal separator disposed along the lead between the electrodes and the at least one terminal; at least one terminal conductor electrically coupling one of the at least one terminal to the signal separator; and multiple electrode conductors. Each electrode conductor electrically couples the signal separator to a different one of the electrodes. The lead has more electrode conductors than terminal conductors. The signal separator is configured and arranged to receive signals from the at least one terminal conductor and to separate the signals by frequency into electrode signals. Each electrode signal is directed along a pre-determined one of the electrode conductors to a corresponding one of the electrodes based on the frequency of the electrode signal.

In another embodiment, an electrical stimulating system includes the electrical stimulation lead described above; an implantable pulse generator coupleable to the electrical stimulation lead; and a connector for receiving the electrical stimulation lead. The implantable pulse generator includes a housing, and an electronic subassembly disposed in the housing. The connector has a proximal end, a distal end, and a longitudinal length. The connector includes a connector housing defining a port at the distal end of the connector. The port is configured and arranged for receiving the proximal end of the lead body of the electrical stimulation lead. The connector also includes at least one connector contact disposed in the connector housing. The at least one connector contact is configured and arranged to couple to the at least one terminal disposed on the proximal end of the lead body of the electrical stimulation lead.

In yet another embodiment, an electrical stimulating system includes an external signal source configured and arranged to be positioned outside of a patient's body; and an implantable lead. The lead includes at least one lead body having a distal end portion, a proximal end portion, and a longitudinal length; multiple electrodes disposed along the distal end portion of the at least one lead body; a signal separator disposed along the lead; at least one proximal conductor; and multiple electrode conductors. Each proximal conductor electrically couples to the signal separator and is configured and arranged to receive signals transmitted by the external signal source; and each electrode conductor electrically couples the signal separator to a different one of the electrodes. The lead has more electrode conductors than proximal conductors. The signal separator is configured and arranged to receive the signals from the at least one proximal conductor and to separate the signals by frequency into electrode signals. Each electrode signal is directed along a pre-determined one of the electrode conductors to a corresponding one of the electrodes based on the frequency of the electrode signal.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic side view of one embodiment of an electrical stimulation system that includes a paddle lead electrically coupled to an implantable pulse generator, according to the invention;

FIG. 2 is a schematic side view of one embodiment of an electrical stimulation system that includes a percutaneous lead electrically coupled to an implantable pulse generator, according to the invention;

FIG. 3A is a schematic side view of one embodiment of the implantable pulse generator of FIG. 1 configured and arranged to electrically couple to an elongated device, according to the invention;

FIG. 3B is a schematic side view of one embodiment of a lead extension configured and arranged to electrically couple the elongated device of FIG. 2 to the implantable pulse generator of FIG. 1, according to the invention;

FIG. 4 is a schematic diagram of one embodiment of an electrical stimulation system, according to the invention;

FIG. 5 is a schematic diagram of a second embodiment of an electrical stimulation system, according to the invention;

FIG. 6 is a schematic diagram of a third embodiment of an electrical stimulation system, according to the invention;

FIG. 7 is a schematic diagram of a distal portion a fourth embodiment of an electrical stimulation system, according to the invention; and

FIG. 8 is a schematic overview of one embodiment of components of a stimulation system, including an electronic subassembly disposed within an implantable pulse generator, according to the invention.

### DETAILED DESCRIPTION

The present invention is directed to the area of implantable electrical stimulation systems and methods of making and using the systems. The present invention is also directed implantable electrical stimulation leads using superposition signals for delivering neuromodulation signals, as well as methods of making and using the leads and electrical stimulation systems.

Suitable implantable electrical stimulation systems include, but are not limited to, a least one lead with one or more electrodes disposed along a distal end of the lead and one or more terminals disposed along the one or more proximal ends of the lead. Leads include, for example, percutaneous leads, paddle leads, and cuff leads. Examples of electrical stimulation systems with leads are found in, for example, U.S. Pat. Nos. 6,181,969; 6,516,227; 6,609,029; 6,609,032; 6,741,892; 7,949,395; 7,244,150; 7,672,734; 7,761,165; 7,974,706; 8,175,710; 8,224,450; and 8,364,278; and U.S. Patent Application Publication No. 2007/0150036, all of which are incorporated by reference.

In many conventional electrical stimulation leads, a large number of electrical conductors extend along the length of the lead. In some leads, there is a conductor for each of the electrodes at the distal end of the lead. The leads described herein utilize fewer conductors (in some embodiments, only a single conductor) along much of the length of the lead to provide independent signals to multiple electrodes by superposition of the signals and later separation near the electrodes. Utilizing fewer conductors along a portion of the lead can result in that portion having a thinner diameter or cross-section.

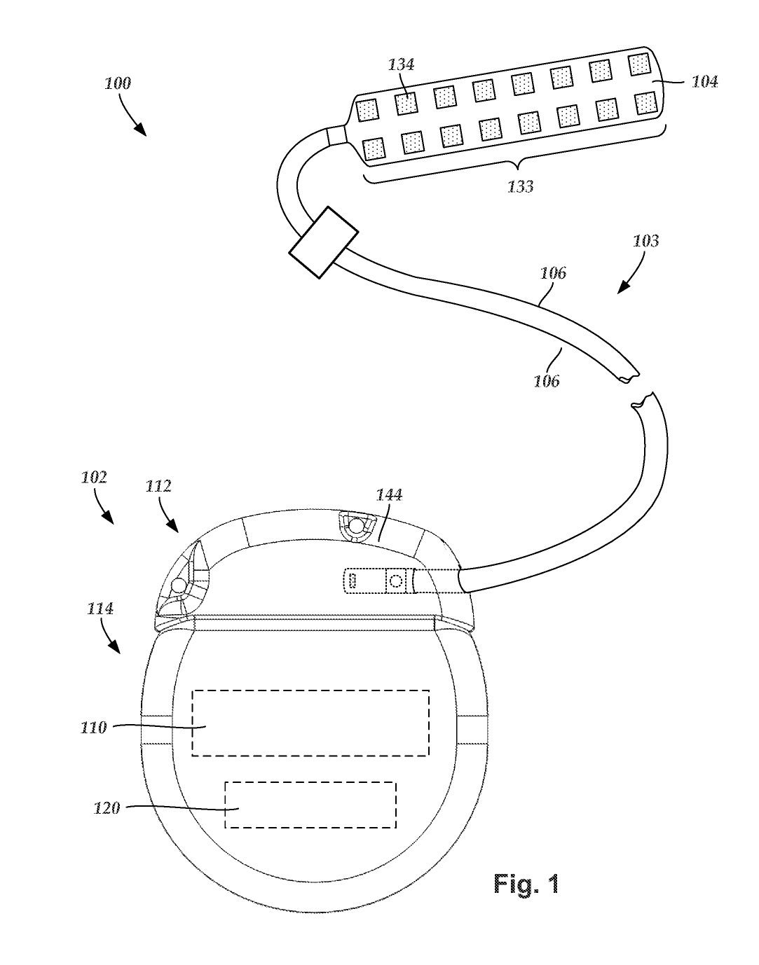

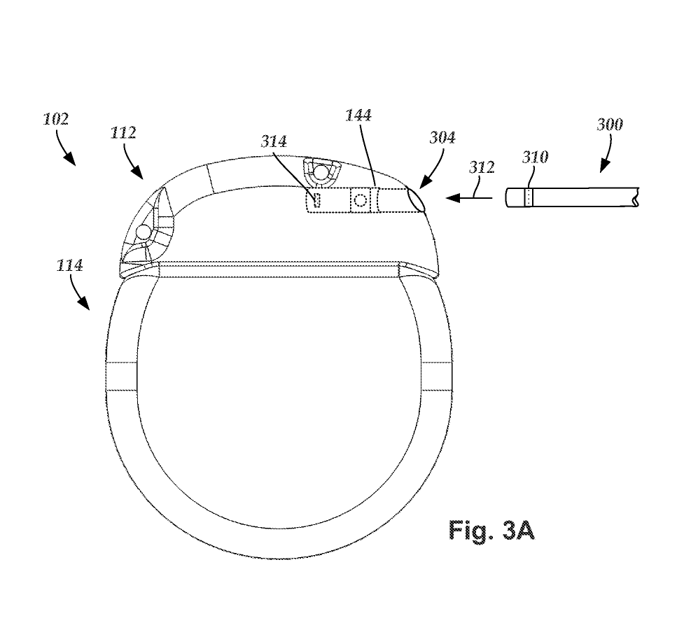

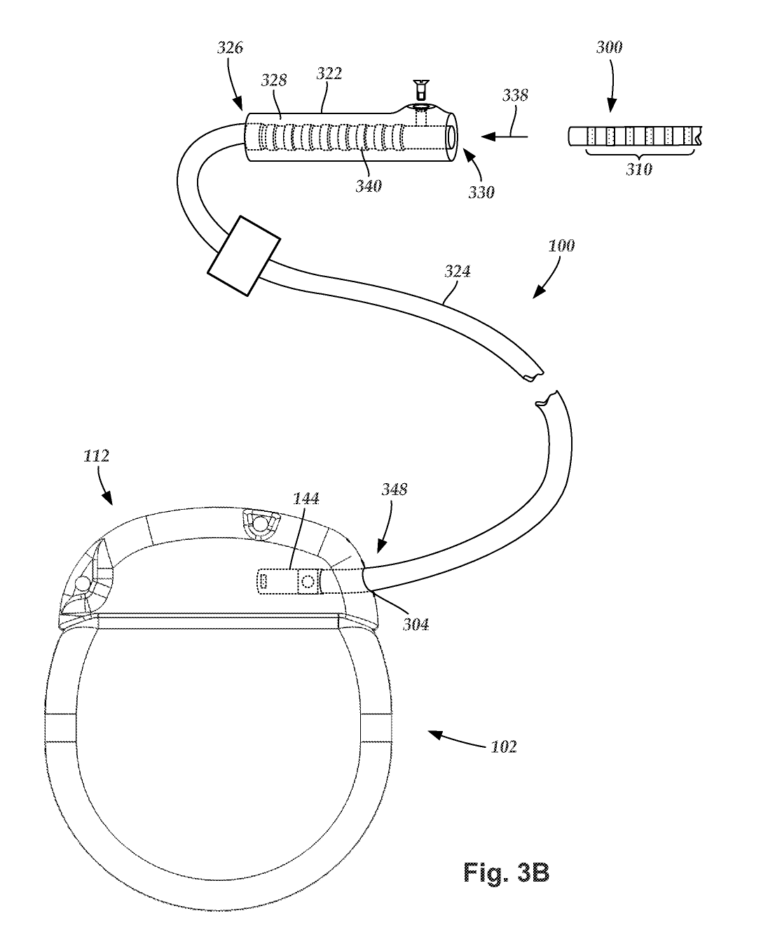

FIG. 1 illustrates schematically one embodiment of an electrical stimulation system 100. The electrical stimulation system includes an implantable pulse generator (e.g., a stimulator or control module) 102 and a lead 103 coupleable to the implantable pulse generator 102. The lead 103 includes a paddle body 104 and one or more lead bodies 106. In FIG. 1, the lead 103 is shown having one lead body 106. It will be understood that the lead 103 can include any suitable number of lead bodies including, for example, one, two, three, four, five, six, seven, eight or more lead bodies 106. An array of electrodes 133, such as electrode 134, is disposed on the paddle body 104, and one or more terminals (e.g., 310 in FIGS. 3A and 3B) are disposed along each of the one or more lead bodies 106. In at least some embodiments, the lead has more electrodes than terminals.

It will be understood that the electrical stimulation system can include more, fewer, or different components and can have a variety of different configurations including those configurations disclosed in the electrical stimulation system references cited herein. For example, instead of a paddle body, the electrodes can be disposed in an array at or near the distal end of a lead body forming a percutaneous lead.

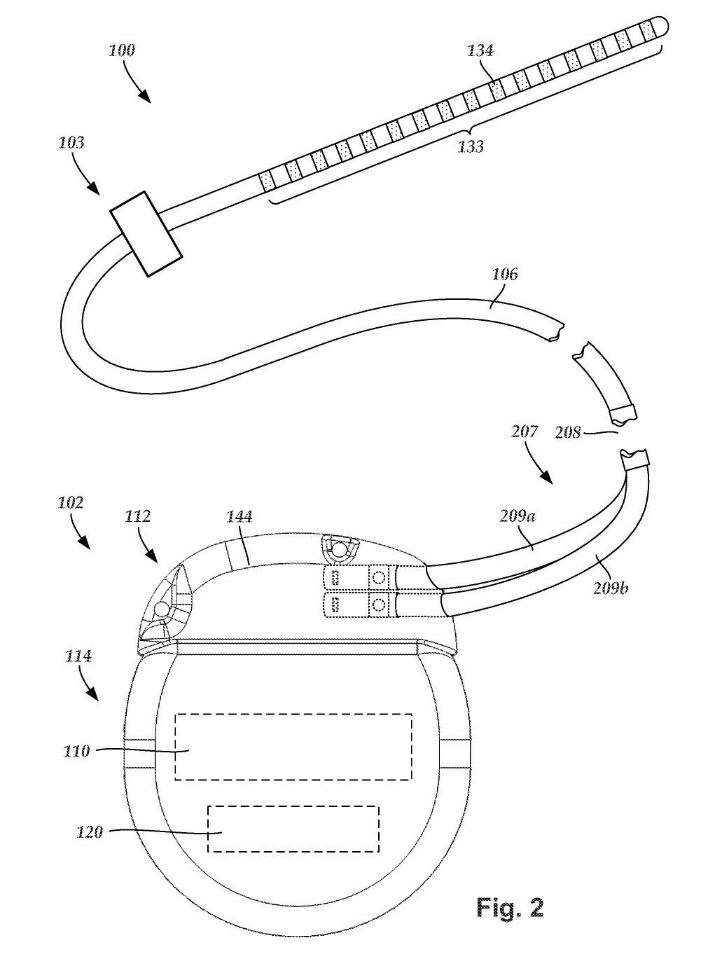

FIG. 2 illustrates schematically another embodiment of the electrical stimulation system 100, where the lead 103 is a percutaneous lead. In FIG. 2, the electrodes 134 are shown disposed along the one or more lead bodies 106. In at least some embodiments, the lead 103 is isodiametric along a longitudinal length of the lead body 106.

The lead 103 can be coupled to the implantable pulse generator 102 in any suitable manner. In FIG. 1, the lead 103 is shown coupling directly to the implantable pulse generator 102. In at least some other embodiments, the lead 103 couples to the implantable pulse generator 102 via one or more intermediate devices (300 in FIGS. 3A and 3B). For example, in at least some embodiments one or more lead extensions 324 (see e.g., FIG. 3B) can be disposed between the lead 103 and the implantable pulse generator 102 to extend the distance between the lead 103 and the implantable pulse generator 102. Other intermediate devices may be used in addition to, or in lieu of, one or more lead extensions including, for example, a splitter, an adaptor, or the like or combinations thereof. It will be understood that, in the case where the electrical stimulation system 100 includes multiple elongated devices disposed between the lead 103 and the implantable pulse generator 102, the intermediate devices may be configured into any suitable arrangement.

In FIG. 2, the electrical stimulation system 100 is shown having a splitter 207 configured and arranged for facilitating coupling of the lead 103 to the implantable pulse generator 102. The splitter 207 includes a splitter connector 208 configured to couple to a proximal end of the lead 103, and one or more splitter tails 209a and 209b configured and arranged to couple to the implantable pulse generator 102 (or another splitter, a lead extension, an adaptor, or the like).

The implantable pulse generator 102 typically includes a connector housing 112 and a sealed electronics housing 114. An electronic subassembly 110 and an optional power source 120 are disposed in the electronics housing 114. A connector 144 is disposed in the connector housing 112. The connector 144 is configured and arranged to make an electrical connection between the lead 103 and the electronic subassembly 110 of the implantable pulse generator 102.

The electrical stimulation system or components of the electrical stimulation system, including the paddle body 104, the one or more of the lead bodies 106, and the implantable pulse generator 102, are typically implanted into the body of a patient. The electrical stimulation system can be used for a variety of applications including, but not limited to deep brain stimulation, neural stimulation, spinal cord stimulation, muscle stimulation, and the like.

The electrodes 134 can be formed using any conductive, biocompatible material. Examples of suitable materials include metals, alloys, conductive polymers, conductive carbon, and the like, as well as combinations thereof. In at least some embodiments, one or more of the electrodes 134 are formed from one or more of: platinum, platinum iridium, palladium, palladium rhodium, or titanium.

Any suitable number of electrodes 134 can be disposed on the lead including, for example, four, five, six, seven, eight, nine, ten, eleven, twelve, fourteen, sixteen, twenty-four, thirty-two, or more electrodes 134. In the case of paddle leads, the electrodes 134 can be disposed on the paddle body 104 in any suitable arrangement. In FIG. 1, the electrodes 134 are arranged into two columns, where each column has eight electrodes 134.

The electrodes of the paddle body 104 (or one or more lead bodies 106) are typically disposed in, or separated by, a non-conductive, biocompatible material such as, for example, silicone, polyurethane, polyetheretherketone (“PEEK”), epoxy, and the like or combinations thereof. The one or more lead bodies 106 and, if applicable, the paddle body 104 may be formed in the desired shape by any process including, for example, molding (including injection molding), casting, and the like. The non-conductive material typically extends from the distal ends of the one or more lead bodies 106 to the proximal end of each of the one or more lead bodies 106.

In the case of paddle leads, the non-conductive material typically extends from the paddle body 104 to the proximal end of each of the one or more lead bodies 106. Additionally, the non-conductive, biocompatible material of the paddle body 104 and the one or more lead bodies 106 may be the same or different. Moreover, the paddle body 104 and the one or more lead bodies 106 may be a unitary structure or can be formed as two separate structures that are permanently or detachably coupled together.

One or more terminals (e.g., 310 in FIGS. 3A-3B) are typically disposed along the proximal end of the one or more lead bodies 106 of the electrical stimulation system 100 (as well as any splitters, lead extensions, adaptors, or the like) for electrical connection to corresponding connector contacts (e.g., 314 in FIGS. 3A-3B). The connector contacts are disposed in connectors (e.g., 144 in FIGS. 1-3B; and 322 FIG. 3B) which, in turn, are disposed on, for example, the implantable pulse generator 102 (or a lead extension, a splitter, an adaptor, or the like). One or more electrically conductive wires, cables, or the like (i.e., “terminal conductors”—not shown) extend from the terminal(s). In at least some embodiments, there is at least one (or exactly one) terminal conductor for each terminal.

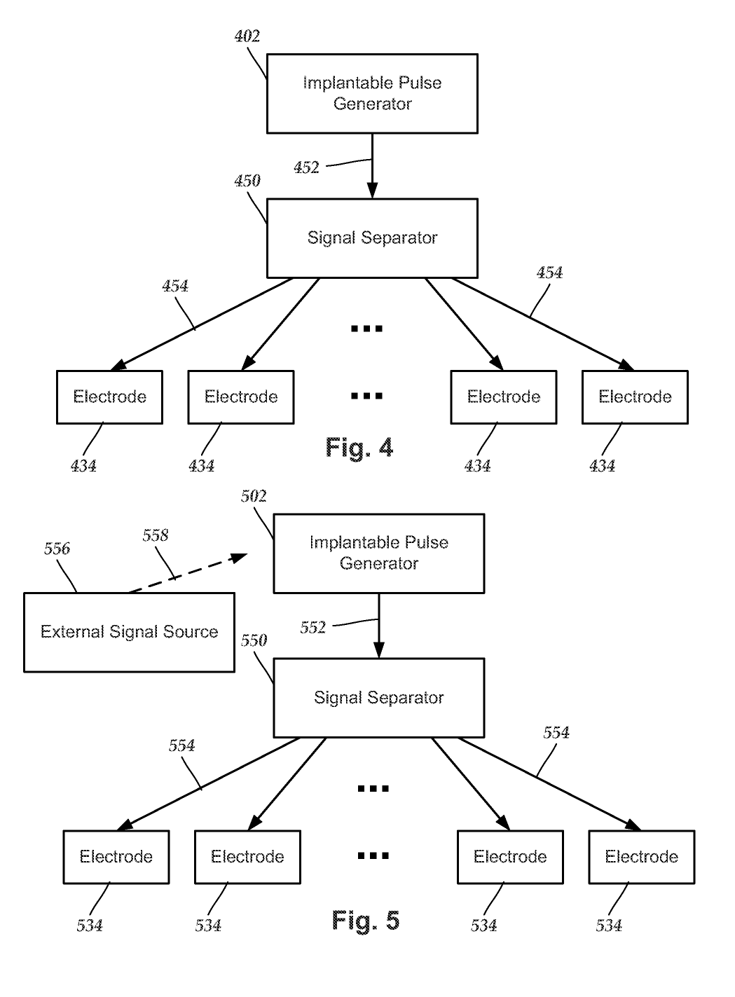

The one or more terminal conductors are embedded in the non-conductive material of the lead body 106 or can be disposed in one or more lumens (not shown) extending along the lead body 106. For example, any of the terminal conductors (identified by reference number 452 in FIG. 4) may extend distally along the lead body 106 from the terminals 310. In some embodiments, there are multiple terminal conductors extending distally from the terminals 310.

FIG. 3A is a schematic side view of one embodiment of a proximal end of one or more elongated devices 300 configured and arranged for coupling to one embodiment of the connector 144. The one or more elongated devices may include, for example, one or more of the lead bodies 106 of FIG. 1, one or more intermediate devices (e.g., a splitter, the lead extension 324 of FIG. 3B, an adaptor, or the like or combinations thereof), or a combination thereof.

The connector 144 defines at least one port into which a proximal end of the elongated device 300 can be inserted, as shown by directional arrow 312. In FIG. 3A (and in other figures), the connector housing 112 is shown having one port 304. The connector housing 112 can define any suitable number of ports including, for example, one, two, three, four, five, six, seven, eight, or more ports.

The connector 144 also includes one or more connector contacts, such as connector contact 314, disposed within each port 304. When the elongated device 300 is inserted into the port 304, the connector contact(s) 314 can be aligned with the terminal(s) 310 disposed along the proximal end(s) of the elongated device(s) 300 to electrically couple the implantable pulse generator 102 to the electrodes (134 of FIG. 1) disposed on the paddle body 104 of the lead 103. Examples of connectors in implantable pulse generators are found in, for example, U.S. Pat. Nos. 7,244,150 and 8,224,450, which are incorporated by reference.

FIG. 3B is a schematic side view of another embodiment of the electrical stimulation system 100. The electrical stimulation system 100 includes a lead extension 324 that is configured and arranged to couple one or more elongated devices 300 (e.g., one of the lead bodies 106 of FIGS. 1 and 2, the splitter 207 of FIG. 2, an adaptor, another lead extension, or the like or combinations thereof) to the implantable pulse generator 102. In FIG. 3B, the lead extension 324 is shown coupled to a single port 304 defined in the connector 144. Additionally, the lead extension 324 is shown configured and arranged to couple to a single elongated device 300. In alternate embodiments, the lead extension 324 is configured and arranged to couple to multiple ports 304 defined in the connector 144, or to receive multiple elongated devices 300, or both.

A lead extension connector 322 is disposed on the lead extension 324. In FIG. 3B, the lead extension connector 322 is shown disposed at a distal end 326 of the lead extension 324. The lead extension connector 322 includes a connector housing 328. The connector housing 328 defines at least one port 330 into which terminal(s) 310 of the elongated device 300 can be inserted, as shown by directional arrow 338. The connector housing 328 also includes a plurality of connector contacts, such as connector contact 340. When the elongated device 300 is inserted into the port 330, the connector contacts 240 disposed in the connector housing 328 can be aligned with the terminal(s) 310 of the elongated device 300 to electrically couple the lead extension 324 to the electrodes (134 of FIGS. 1 and 2) disposed along the lead (103 in FIGS. 1 and 2).

In at least some embodiments, the proximal end of the lead extension 324 is similarly configured and arranged as a proximal end of the lead 103 (or other elongated device 300). The lead extension 324 may include one or more electrically conductive wires (not shown) that electrically couple the connector contact(s) 340 to a proximal end 348 of the lead extension 324 that is opposite to the distal end 326. The conductive wire(s) disposed in the lead extension 324 can be electrically coupled to one or more terminals (not shown) disposed along the proximal end 348 of the lead extension 324. The proximal end 348 of the lead extension 324 is configured and arranged for insertion into a connector disposed in another lead extension (or another intermediate device). As shown in FIG. 3B, the proximal end 348 of the lead extension 324 is configured and arranged for insertion into the connector 144.

Signals to multiple independent electrodes can be directed along a single terminal conductor by superposition (e.g., multiplexing) of the signals. For example, each independent electrode may be associated with a particular frequency or frequency range. Signals for that electrode are generated with that frequency or frequency range and directed along the terminal conductor. Further down the lead (for example, near the distal end of the lead) is a signal separator that separates (e.g., demultiplexes) the signals for the individual electrodes and sends each individual signal along the appropriate electrode conductor to the designated electrode.

FIG. 4 is a schematic block/flow diagram of one embodiment of an electrical stimulation system. The electrical stimulation system, such as the system 100 shown in FIGS. 1, 2 and 3B, includes an implantable pulse generator (IPG) 402, a signal separator 450, and electrodes 434, which are located on one or more electrical stimulation leads, such as lead 103 discussed above. The IPG 402 is electrically coupled to the signal separator 450 through a single terminal conductor 452 which extends distally from a lead terminal. In other embodiments, the lead 103 includes multiple terminals and multiple terminal conductors 452 that extend to the signal separator 450. Typically, however, there are fewer terminal conductors 452 than electrodes 434. Each terminal conductor 452 is capable of carrying superposition (e.g., multiplexed) signals to the signal separator 450.

The structure and function of IPG 402 can be similar to that of IPG 102. In at least some embodiments, the IPG 402 generates pulses of stimulation energy at a low frequency pulse rate (for example, in a range from 1 Hz to 100 KHz). The IPG 402 is capable of generating such pulses for any of the electrodes. To accomplish the superposition of signals, the energy pulses have an underlying AC signal which has a high frequency (for example, in the 200 KHz to 1 GHz range). The signal for each electrode is associated with a different frequency or frequency range which are spaced apart sufficient to be reliably separated by the signal separator 450. It will be understood that the signals can be multiplexed using signal characteristics other than frequency according to know multiplexing methods.

The IPG 402 combines the signals of different frequencies for generating the superposition (e.g., multiplexed) signals. It is to be noted that the pulse rate of the energy pulses is neither used for multiplexing the signals of different frequencies nor separating (discussed later) these signals. The IPG 402 delivers the superposition (e.g., multiplexed) signals in the form of a pulsed electrical waveform to the signal separator 450 via the terminal conductor(s) 452. If there are multiple terminal conductors, there may be multiple superposition signals. Moreover, each superposition signal contains signals for one or more electrodes depending on the desired stimulation pattern.

The IPG 402 generates the electrical stimulation signals using a set of modulation parameters. Examples of these modulation parameters include, but are not limited to, electrode combinations, which define the electrodes that are activated as anodes (positive), cathodes (negative), and turned off (zero), percentage of modulation energy assigned to each electrode, and electrical pulse parameters, which define the pulse amplitude (measured in milliamps or volts depending on whether the IPG supplies constant current or constant voltage to the array of electrodes), pulse width (measured in microseconds), and burst rate (measured as the modulation ON duration X and modulation OFF duration Y). Each signal for each electrode can have a different set of modulation parameters.

The signal separator 450 receives the superposition (e.g., multiplexed) signal(s) through the terminal conductor(s) 452 and separates the superposition signal(s) into individual electrode signals. For example, the signal separator 450 may separate the individual electrode signals by frequency. The signal separator 450 is electrically coupled to each of the electrodes 434 through respective electrode conductors 454. The signal separator 450 may be a single device or may be distributed into multiple devices.

The signal separator 450 can be positioned anywhere along the lead. For example, in some embodiments of paddle leads, the signal separator is disposed proximal to the paddle, or within the paddle body 104, at the distal portion of the lead 103. In some embodiments of isodiametric leads, the signal separator is disposed proximal to all of the electrodes, but along the distal end portion of the lead. In some embodiments, the signal separator 450 may be disposed adjacent to the electrodes 434. In some embodiments, placement of the signal separator 450 is at or along the distal end portion of the lead 103, and thus the signal separator 450 can be accommodated without major modifications to the lead 103. In some embodiments, the signal separator may be distributed so that there is a portion adjacent to each electrode (or to sets of electrodes) to separate out the signal for that electrode (or set of electrodes).

In some embodiments, the signal separator 450 may be disposed partway along the lead 103 so that the lead 103 is narrow along one portion and then wider distal to the signal separator 450. Accordingly the narrow portion of the lead 103 may facilitate placement of lead 103 within a relatively tight region in the patient's body. Further, the number of terminal conductors 452 to be embedded into the lead body 104 is reduced, thereby decreasing complexity of the lead forming process, based on the above placement of the signal separator 450 and the fact that the multiplexed signals are fed to the signal separator 450 via a single (or only a few) terminal conductor(s) 452.

The signal separator 450 can include any suitable components for separating the individual signals. For example, the signal separator 450 can include multiple frequency filters, such as band pass filters, high pass filters, or low pass filters, or any combination thereof, to separate or demultiplex and pass the individual electrode signals based on signal frequency. In at least some embodiments, the frequency filters include only passive elements, such as inductors, resistors, and capacitors. In some embodiments, some or all of these elements can be formed as an integrated circuit. Alternatively or additionally, some or all of the elements can be discrete components. In some embodiments, each frequency filter is disposed in a dedicated manner adjacent to the corresponding electrode 434.

FIG. 5 is a schematic diagram of another embodiment of an electrical stimulation system that includes an IPG 502 electrically coupled through one or more terminal conductors 552 to a signal separator 550, which in turn is electrically coupled to the electrodes 534 via electrode conductors 554. In this embodiment, the signals are transmitted (arrow 558) to the IPG 502 by an external signal source 556 and so the IPG 502 may require fewer components and may be cheaper to produce and smaller in size. In some embodiments, the IPG 502 does not need to include a battery or a processor and may be limited to including only passive circuit elements The signal separator 550 and electrodes 534 have similar structure and operate in a manner similar to that discussed above for signal separator 450 and electrodes 434 respectively.

The external signal source 556 can wirelessly transmit superposition (e.g., multiplexed) signals 558 to the IPG 502. In order to transmit the signals, the external signal source 556 is operatively connected to an external power supply (not shown), and includes an antenna and a processor. The antenna may include, for example, tuning circuitry and a single coil or a group of coils, each coil having one or more turns and collectively arranged for either uni-directional or omni-directional wireless transfer of multiplexed signals or energy 558.

The processor of the external signal source 556 can be, for example, any microcontroller-based electro-mechanical device, or a computing device, or any combination of such devices. The processor may be configured to perform different tasks. For example, the processor may be configured to detect the presence of an antenna located in the IPG 502 as well as to control electrical parameters, such as frequency, amplitude, and/or power of the energy in the antenna. Accordingly, the external signal source 556 may radiate wireless superposition (e.g., multiplexed) signals 558, which may be received by an antenna (e.g., a coil or a tuned coil) in the IPG 502.

The IPG 502 delivers the wirelessly received superposition signals 558 to the signal separator 550 via one or more terminal conductors 552. The signal separator 550 then separates (e.g., demultiplexes) the superposition (e.g., multiplexed) signals 558 by frequency into electrode signals, each of which is fed to the electrodes 534 via the respective electrode conductors 554.

In some embodiments, the external signal source 556 wirelessly transmits various AC electrical signals 558 of different frequencies to the IPG 502 and the IPG 502 then combines these AC electrical signals to form the superposition (e.g., multiplexed) signal that is delivered to the signal separator 550.

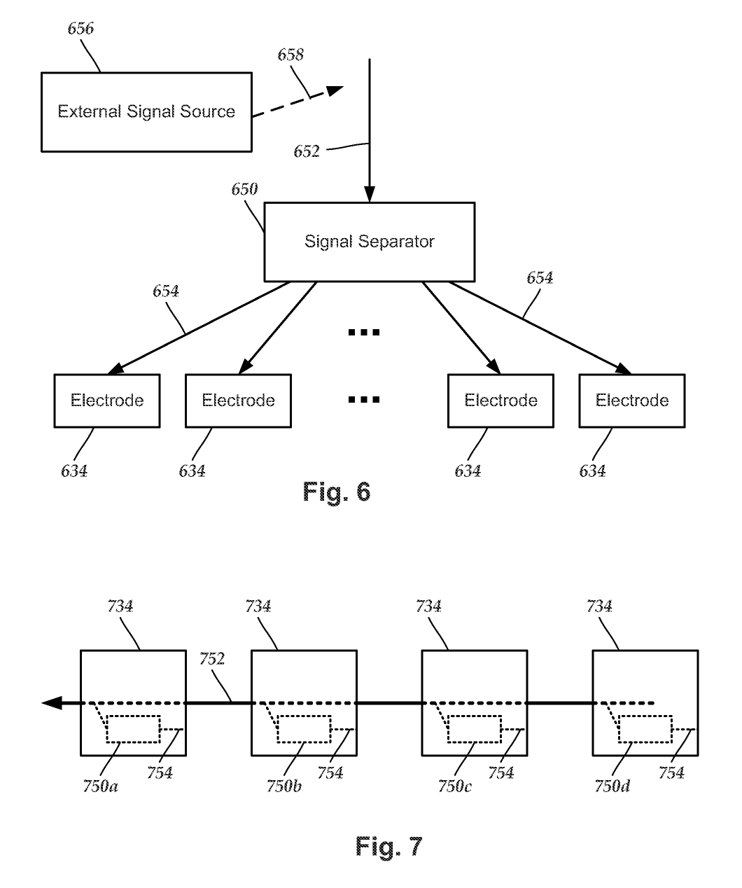

FIG. 6 is a schematic diagram of another embodiment of an electrical stimulation system, but this system does not include an IPG. The electrical stimulation system includes a signal separator 650 proximally coupled to a proximal conductor 652 and distally coupled to electrodes 634 via electrode conductors 654. The structure, function and position of the signal separator 650, electrodes 634, and electrode conductors 654 is similar to that discussed above for signal separator 450, electrodes 434, and electrode conductors 454.

The electrical stimulation system may further include an external signal source 656 that has a structure and function similar to that discussed with regard to external signal source 556. The external signal source 656 can be an external IPG and can be single device or multiple connected devices. In some embodiments, the external signal source can be separated into two modules connected by any suitable device, such as a cable, or in wireless communication. The first module may include the bulk of electronics, such as the processor, power source, and signal generator, and may be worn by a user, such as on the user's belt, or may be located at or on any other external structure. The second module may include an antenna which may be externally positioned, for example, over the proximal conductor or an antenna associated with the proximal conductor to facilitate reliable communication between eh external single source and the lead. External/internal magnets may be used in these embodiments to hold the second module in place with respect to the lead.

The proximal conductor 652 acts as an antenna, which may be tuned using passive elements, to receive signals from the external signal source 656. In some embodiments, the proximal conductor 652 includes an antenna coil, although any wire may act as an antenna. In order to perform this operation, the antenna of the external signal source 656 and the proximal conductor 652 may be designed to have a quality factor so that the coils inductively couple to each other in a reliable manner. The quality factor, referred to as the Q factor, may be defined as a ratio of the apparent power to the power losses in a coil:

$$Q = \frac{{ω}_{o} \cdot L}{R} = \frac{Apparent Power}{Power Losses} \qquad ( 1 )$$

where:
   ωₒ=2πf
   f=Operating frequency of electrical signal in a coil
   L=Inductance of the coil
   R=Effective resistance of the coil

The quality factor of a coil may determine, and in some cases largely determines, the degree of frequency selectivity of a tuned circuit that may be achieved using a coil and a tuning element, such as the capacitor. The Q factor may be enhanced or otherwise improved using any known methods.

FIG. 7 is a schematic diagram of a one embodiment of a distributed signal separator for an electrical stimulation system that includes electrodes 734. The signal separator is distributed into separate signal separator elements 750a, 750b, 750c, and 750d that is associated with, and disposed adjacent to or beneath, a corresponding electrode. The individual signal separator elements is coupled to a terminal conductor 752 and is electrically coupled to its corresponding electrode 734 via an electrode conductor 754. The signal separators 750 may receive the multiplexed signals from an IPG (as discussed with regard to IPG 402 in FIG. 4 and IPG 502 in FIG. 5), or an external signal source (as discussed with regard to external signal source 556 in FIG. 5 and external signal source 656 in FIG. 6). The electrodes 734 may be serially disposed at or along the distal portion of a terminal conductor 752, which is proximally coupled to either the IPG 402, 502 via one or more terminals, such as the terminal 310, or the proximal conductor 652.

The arrangements described herein may provide one or more of the following benefits: 1) reducing manufacturing complexity of the lead, 2) providing a broader level of therapy for treatment of different diseases and disorders, and/or 3) reducing the number of electronic components so as to reduce stimulator cost and size, while enhancing stimulator reliability or simplifying the therapeutic procedure.

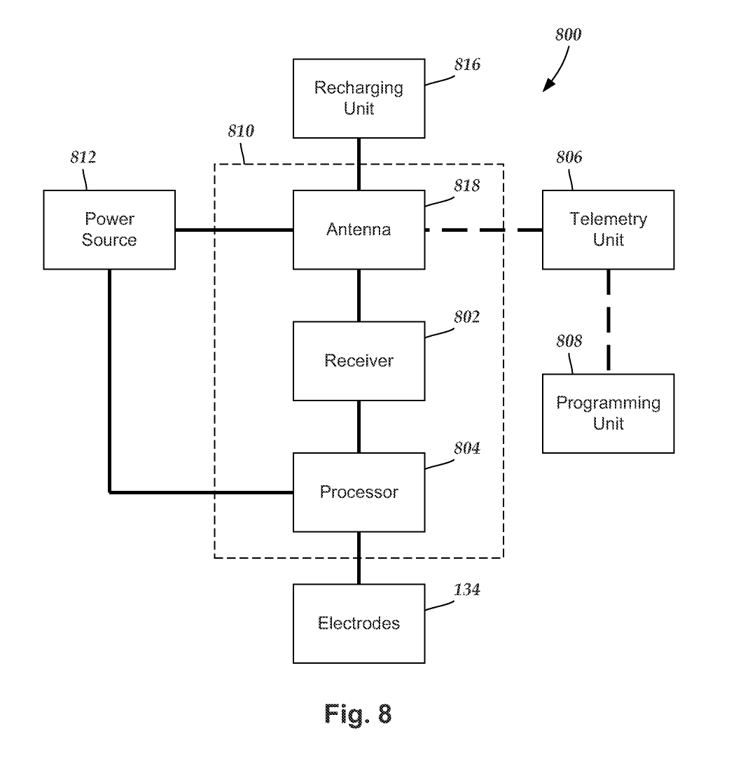

FIG. 8 is a schematic overview of one embodiment of components of an electrical stimulation system 800 including an electronic subassembly 810, which may be disposed within an implantable pulse generator or an external signal source. It will be understood that the electrical stimulation system can include more, fewer, or different components and can have a variety of different configurations including those configurations disclosed in the stimulator references cited herein.

In some embodiments similar to those discussed in the description of FIGS. 4 and 5, some of the components (for example, a power source 812, an antenna 818, a receiver 802, and a processor 804) of the electrical stimulation system can be positioned on one or more circuit boards or similar carriers within a sealed housing of an implantable pulse generator. In some other embodiments similar to those discussed in the description of FIGS. 6 and 7, these components of the electrical stimulation system are positioned on one or more circuit boards or similar within an external signal source such as 556, 656. Any power source 812 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bio-energy power sources, fuel cells, bioelectric cells, osmotic pressure pumps, and the like including the power sources described in U.S. Pat. No. 7,437,193, incorporated herein by reference.

As another alternative, power can be supplied by an external power source 812 through inductive coupling via the optional antenna 818 or a secondary antenna. The external power source can be in a device that is mounted on the skin of the user or in a unit that is provided near the user on a permanent or periodic basis.

If the power source 812 is a rechargeable battery, the battery may be recharged using the optional antenna 818, if desired. Power can be provided to the battery for recharging by inductively coupling the battery through the antenna to a recharging unit 816 external to the user. Examples of such arrangements can be found in the references identified above.

In one embodiment, the processor 804 may be configured to combine various electrical signals of different frequencies to produce multiplexed signals, which are demultiplexed based on frequency into electrode signals by a signal separator, such as signal separator 450 shown in FIG. 4. Alternatively, the multiplexed signals may be received by the antenna 818 from the telemetry unit 806. The signal separator may be embedded into the receiver 802 and electrically coupled to the electrodes 134.

Upon receiving the electrode signals, electrical current is emitted by the electrodes 134 on the paddle or lead body to stimulate nerve fibers, muscle fibers, or other body tissues near the electrical stimulation system. The processor 804 is generally included to control the timing and electrical characteristics of the electrical stimulation system. For example, the processor 804 can, if desired, control one or more of the timing, frequency, strength, duration, and waveform of the pulses. In addition, the processor 804 can select which electrodes can be used to provide stimulation, if desired. In some embodiments, the processor 804 selects which electrode(s) are cathodes and which electrode(s) are anodes. In some embodiments, the processor 804 is used to identify which electrodes provide the most useful stimulation of the desired tissue.

Any processor can be used and can be as simple as an electronic device that, for example, produces pulses at a regular interval or the processor can be capable of receiving and interpreting instructions from an external programming unit 808 that, for example, allows modification of pulse characteristics. In the illustrated embodiment, the processor 804 is coupled to a receiver 802 which, in turn, is coupled to the optional antenna 818. This allows the processor 804 to receive instructions from an external source to, for example, direct the pulse characteristics and the selection of electrodes, if desired.

In one embodiment, the antenna 818 is capable of receiving signals (e.g., RF signals) from an external telemetry unit 806 which is programmed by the programming unit 808. The programming unit 808 can be external to, or part of, the telemetry unit 806. The telemetry unit 806 can be a device that is worn on the skin of the user or can be carried by the user and can have a form similar to a pager, cellular phone, or remote control, if desired. As another alternative, the telemetry unit 806 may not be worn or carried by the user but may only be available at a home station or at a clinician's office. The programming unit 808 can be any unit that can provide information to the telemetry unit 806 for transmission of wireless multiplexed signals to the electrical stimulation system 800. The programming unit 808 can be part of the telemetry unit 806 or can provide signals or information to the telemetry unit 806 via a wireless or wired connection. One example of a suitable programming unit is a computer operated by the user or clinician to send signals to the telemetry unit 806.

In another embodiment, the electronic subassembly 810 of electrical stimulation system 800 may not include the receiver 802 and the processor 804. The telemetry unit 806 may wirelessly transmit multiplexed signals to the antenna 818, which also behaves as a receiver.

The signals sent to the processor 804 via the antenna 818 and the receiver 802 can be used to modify or otherwise direct the operation of the electrical stimulation system. For example, the signals may be used to modify the pulses of the electrical stimulation system such as modifying one or more of pulse duration, pulse frequency, pulse waveform, and pulse strength. The signals may also direct the electrical stimulation system 800 to cease operation, to start operation, to start charging the battery, or to stop charging the battery. In other embodiments, the stimulation system does not include the antenna 818 or receiver 802 and the processor 804 operates as programmed.

Optionally, the electrical stimulation system 800 may include a transmitter (not shown) coupled to the processor 804 and the antenna 818 for transmitting signals back to the telemetry unit 806 or another unit capable of receiving the signals. For example, the electrical stimulation system 800 may transmit signals indicating whether the electrical stimulation system 800 is operating properly or not or indicating when the battery needs to be charged or the level of charge remaining in the battery. The processor 804 may also be capable of transmitting information about the pulse characteristics so that a user or clinician can determine or verify the characteristics.

In some embodiments, passive elements constitute the antenna 818 and the receiver 802 which are part of the lead 103. In these embodiment, the processor 804 is not included in the lead, instead functionality of the processor 804 is transferred to the programming unit 808, which is configured to generate the superposition (e.g., multiplexed) signals.

The above specification and examples provide a description of the manufacture and use of the invention. Since many embodiments of the invention can be made without departing from the spirit and scope of the invention, the invention also resides in the claims hereinafter appended.

What is claimed as new and desired to be protected by Letters Patent of the United States is:

1. An electrical stimulation lead, comprising:
at least one lead body having a distal end portion, a proximal end portion, and a longitudinal length;
a plurality of electrodes disposed along the distal end portion of the at least one lead body;
at least one terminal disposed along the proximal end portion of the at least one lead body, wherein the lead has more electrodes than terminals;
a signal separator disposed along the lead between the plurality of electrodes and the at least one terminal;
at least one terminal conductor, each terminal conductor electrically coupling a one of the at least one terminal to the signal separator; and
a plurality of electrode conductors, each electrode conductor electrically coupling the signal separator to a different one of the plurality of electrodes, wherein the lead has more electrode conductors than terminal conductors;
wherein the signal separator is configured and arranged to receive signals from the at least one terminal conductor and to separate the signals by frequency into electrode signals, wherein each electrode signal is directed along a pre-determined one of the electrode conductors to a corresponding one of the electrodes based on the frequency of the electrode signal.

2. The lead of claim 1, wherein the distal end portion of the at least one lead body comprises a paddle, wherein the plurality of electrodes are disposed on the paddle.

3. The lead of claim 2, wherein the signal separator is disposed in the paddle.

4. The lead of claim 2, wherein the signal separator is disposed proximal to the paddle.

5. The lead of claim 1, wherein the distal end portion of each of the at least one lead body is isodiametric.

6. The lead of claim 1, wherein the signal separator comprises a plurality of frequency filters configured and arranged to separate the signals by frequency into the electrode signals.

7. The lead of claim 6, wherein each frequency filter is disposed adjacent a different one of the electrodes.

8. The lead of claim 6, wherein each frequency filter is a band pass filter and each band pass filter is configured and arranged to pass a different frequency range.

9. The lead of claim 1, wherein the signal separator is disposed along the distal end portion of the lead.

10. An electrical stimulating system, comprising:
the electrical stimulation lead of claim 1;
an implantable pulse generator coupleable to the electrical stimulation lead, the implantable pulse generator comprising
a housing, and
an electronic subassembly disposed in the housing; and
a connector for receiving the electrical stimulation lead, the connector having a proximal end, a distal end, and a longitudinal length, the connector comprising
a connector housing defining a port at the distal end of the connector, the port configured and arranged for receiving the proximal end of the lead body of the electrical stimulation lead, and
at least one connector contact disposed in the connector housing, the at least one connector contact configured and arranged to couple to the at least one terminal disposed on the proximal end of the lead body of the electrical stimulation lead.

11. The system of claim 10, further comprising an external signal source configured and arranged to transmit signals to the implantable pulse generator.

12. An electrical stimulating system, comprising:
an external signal source configured and arranged to be positioned outside of a patient's body; and
an implantable lead comprising
at least one lead body having a distal end portion, a proximal end portion, and a longitudinal length;
a plurality of electrodes disposed along the distal end portion of the at least one lead body;
a signal separator disposed along the lead;
at least one proximal conductor, each proximal conductor electrically coupled to the signal separator and configured and arranged to receive signals transmitted by the external signal source; and
a plurality of electrode conductors, each electrode conductor electrically coupling the signal separator to a different one of the plurality of electrodes, wherein the lead comprises more electrode conductors than proximal conductors;
wherein the signal separator is configured and arranged to receive the signals from the at least one proximal conductor and to separate the signals by frequency into electrode signals, wherein each electrode signal is directed along a pre-determined one of the electrode conductors to a corresponding one of the electrodes based on the frequency of the electrode signal.

13. The system of claim 12, wherein the distal end portion of the at least one lead body comprises a paddle, wherein the plurality of electrodes are disposed on the paddle.

14. The system of claim 13, wherein the signal separator is disposed in the paddle.

15. The system of claim 13, wherein the signal separator is disposed proximal to the paddle.

16. The system of claim 12, wherein the distal end portion of each of the at least one lead body is isodiametric.

17. The system of claim 12, wherein the signal separator comprises a plurality of frequency filters configured and arranged to separate the signals by frequency into the electrode signals.

18. The system of claim 17, wherein each frequency filter is disposed adjacent a different one of the electrodes.

19. The system of claim 17, wherein each frequency filter is a band pass filter and each band pass filter is configured and arranged to pass a different frequency range.

20. The system of claim 12, wherein the signal separator is disposed along the distal end portion of the lead.
